(19) United States
(12) Patent Application Publication
(10) Pub. No.: US 2025/0017576 A1
(43) Pub. Date: Jan. 16, 2025

(54) MEDICAL ATTACHMENT ADAPTER FOR RELEASABLY ATTACHING TO A MEDICAL ROBOT, MEDICAL ATTACHMENT SYSTEM AND MEDICAL DEVICE

(71) Applicant: B. Braun New Ventures GmbH, Freiburg (DE)

(72) Inventors: Lukas Hahn, Sigmaringen (DE); John Gray-Dreizler, Niedereschach (GB); Jan Dahmen, Seitingen-Oberflacht (DE); Markus Nesper, Tuttlingen (DE); Pedro Morales, Tuttlingen (ES)

(21) Appl. No.: 18/771,280

(22) Filed: Jul. 12, 2024

(30) Foreign Application Priority Data
Jul. 13, 2023 (DE) 10 2023 118 545.4

(51) Int. Cl. A61B 17/00; A61B 34/30

(52) U.S. Cl. CPC A61B 17/00; A61B 34/30; A61B 2017/00486

(57) ABSTRACT

A medical fastening adapter is detachably fastenable to a fastening section of a medical robot arranged within a sterile cover or barrier. The fastening adapter includes a support structure with an instrument interface. The fastening adapter also includes at least a first, a second, and a third geometric coupling structure designed for positive connection to the fastening section. An adjusting element is operable to change positions of at least the first, second, and third geometric coupling structures relative to one another so that in a first state, the fastening adapter is couplable to the fastening section, and in a second state, the fastening adapter is non-detachably fixed to the fastening section, such that the position of the fastening adapter is fixed relative to the fastening section via the first, second, and third coupling structures. A medical fastening system and a medical device can include the medical fastening adapter.

### CROSS-REFERENCE TO RELATED APPLICATION

This application claims priority under 35 U.S.C. § 119 to German Application No. 10 2023 118 545.4, filed on Jul. 13, 2023, the content of which is incorporated by reference herein in its entirety.

### FIELD

The present disclosure relates to a medical fastening adapter for detachable fastening to a fastening section of a medical robot, in particular a robot-guided surgical microscope, which is arranged in particular within a sterile cover. The medical fastening adapter has a support structure (as a basic structure) with an instrument interface/coupling interface that is adapted (by means of the predefined instrument interface) to couple or uncouple a sterile medical product, such as an instrument or another medical device. In addition, the present disclosure relates to a medical fastening system and a medical device with a sterile cover for fastening sterile medical products to the medical device.

### BACKGROUND

In surgical procedures, medical robots, in particular medical surgical microscopes, are usually used in combination with other medical products such as surgical instruments or an additional endoscope or camera. Accordingly, it is advantageous if the medical robots can be extended by the respective medical products, in particular the surgical instruments, via a coupling, depending on the desired requirements. This requires that the sterile instruments do not come into direct contact with the non-sterile medical robots. A sterile barrier, e.g., in the form of a sterile cover, is usually placed between the instruments to be coupled and the medical robot.

In the state of the art, there are approaches that make it possible to fasten a sterile medical instrument to a medical robot, e.g., a surgical microscope. Traditionally, intended sterile barriers are impaired or even destroyed by this fastening maneuver, which is why in normal cases only a single fastening with subsequent single use of the respective sterile barrier is possible. This means that a medical instrument cannot be re-fastened to the robot without additional intermediate steps. Furthermore, approaches exist that allow multiple changes or adjustments to the orientation of medical instruments attached to a medical robot, but the precision and repeatability of mounting medical instruments over a sterile barrier of these conventional approaches are severely limited.

It is very important that the sterile instruments can be easily and efficiently coupled to the medical robots and that the sterile barrier is not penetrated by coupling processes and, in particular, by recoupling maneuvers.

### SUMMARY

Therefore, the present disclosure is based on the task of avoiding or at least mitigating the disadvantages described above and, in particular, providing a medical fastening adapter, a medical fastening system, and a medical device, in particular a medical robot, for detachably fastening a sterile medical instrument to a medical robot, with which a stable and precise connection between the components (medical products) to be coupled can be produced. Furthermore, a partial task can be seen in designing the fastening adapter or the fastening system in such a way that a sterile barrier arranged between the medical robot and the sterile instrument to be coupled, in particular in the form of a sterile (plastic) cover, remains intact or is not damaged by repeated coupling processes.

Accordingly, the disclosure relates to a medical fastening adapter for detachable fastening to a fastening section of a medical robot, in particular a robot-guided surgical microscope, which (i.e., the medical robot) is arranged in particular within a sterile cover as a sterile barrier, wherein the medical fastening adapter has a rigid support structure (as a type of base frame) with an instrument interface. The medical fastening adapter further has at least a first geometric coupling structure, a second geometric coupling structure, and a third geometric coupling structure, each of which is adapted and designed for a positive connection to the fastening section, and an adjusting element/means/fixing means/closure means which is adapted for this purpose to change the relative spatial position of the at least first, second, and third geometric coupling structure to one another in such a way that in a first state of a first relative positioning the fastening adapter can be coupled to and uncoupled from the fastening section and in a second state of a second relative positioning it is permanently fixed to the fastening section/is fastened/fixed, and by means of the first, second, and third coupling structure determines a defined/unique position of the fastening adapter relative to the fastening section. For example, the first, second, and third coupling structures can point radially inwards in the direction of a fastening section and be spaced further apart in a first relative positioning than in the second relative positioning, wherein in the first relative positioning there is still a distance with a gap (i.e., still without rigid contact) between the coupling structures and the fastening section, so that the fastening adapter is freely movable and can be aligned or arranged and in the second relative positioning the coupling structures are moved towards each other, shortening a distance as a result, a geometric contact is established with corresponding fixation as a result.

In other words, the fastening adapter comprises at least three geometric coupling structures/elements (for uniquely defining a position, similar to mathematics in which a plane can be defined by three points) intended and designed to couple/mount/fasten the fastening adapter to a fastening section formed/arranged on the medical robot. The coupling structures are preferably arranged within a common plane on the support structure. The fastening adapter (for fixing and releasing) has an adjusting means/adjusting element or fixing means, by actuating which the relative position of the geometric coupling structures to one another can be changed/influenced, in particular manually. Actuating the adjusting means moves the fastening adapter from a first state of a first relative positioning of the geometric coupling structures to one another, in which the fastening adapter can be coupled to or uncoupled from the fastening section of the robot, to a second state of a second relative positioning of the geometric coupling structures to one another, in which the fastening adapter is fixed non-detachably to the fastening section. This means that in this second state, the coupling structures of the fastening adapter are aligned with one another in such a way that the fastening adapter is (positively) fastened to the fastening section via these coupling structures, in particular clamps or clasps around it, so that it is fixed to the fastening section of the robot (i.e., the position of the fastening adapter on the fastening section is fixed) without the need to actuate the adjusting means again to release it. Thus, in this second state, a clear position of the fastening adapter relative to the fastening section of the robot is defined or there are no degrees of freedom for a relative movement of these two coupled components to each other. Preferably, the medical fastening adapter has an (instrument) interface to which the medical product, in particular a sterile medical product such as an instrument, that is ultimately to be fastened can be mounted/attached. The fastening adapter thus serves as a (serial) intermediate link for the medical product and the medical robot, wherein the fastening adapter is designed in particular to be sterilizable and/or sterilized in order to be connected in the sterile area.

Advantageously, such a fastening adapter enables a sterile instrument to be connected to a medical robot in such a way that a stable and defined, in particular releasable, coupling of the fastening adapter, and thus of the medical instrument mounted thereon with the medical robot is provided by actuating an adjusting element and the resulting relative movement of specially designed coupling structures of the fastening adapter with respect to one another.

In a first advantageous embodiment, the first coupling structure, the second coupling structure, and the third coupling structure lie in one plane and each have a protruding/projecting convex surface, in particular as a hemisphere, in order to engage positively in a complementary counter-coupling structure, for example as a latching lug. At least the convex surface of the first coupling structure faces the convex surfaces of the second and/or third coupling structure and the convex surface of the first coupling structure is translationally/axially displaceable by means of the adjusting means in order to change a relative distance to the second and/or third coupling structure. The convex surface, particularly in the form of the hemisphere, therefore provides a rounded coupling structure without sharp edges or points, which cannot damage a sterile cover if it is clamped between the fastening adapter and the fastening structure. In other words, the fastening adapter has three coupling structures, each of which is arranged in a common plane and each of which has a convex/curved/partially spherical geometry protruding from the support structure, in particular in the form of a hemisphere. The coupling structures are arranged relative to one another in such a way that a first of the coupling structures or its protruding geometry faces the second and/or the third coupling structure. Preferably, the second and third coupling structures point in the same direction and are arranged parallel to each other. Furthermore, the first coupling structure is axially displaceable by manual actuation of the adjusting means with which the first coupling structure is connected, namely in an axial relative movement towards or away from the second and third coupling structures, whereby the axial relative distance between the first coupling structure and the remaining coupling structures can be changed. This type of design with convex, in particular hemispherical, coupling structures allows the fastening adapter to be aligned with a high degree of precision and at the same time blocks the degrees of freedom of the fastening adapter relative to the medical robot in a coupled state. Advantageously, such a curved design of the coupling structures as contact points does not have any pointed or angular geometries, which could potentially damage/break through a sterile cover/barrier arranged between the contact points of the fastening adapter with the medical robot.

In a further preferred embodiment of the disclosure, the second coupling structure and the third coupling structure, in particular with their convex surfaces, are arranged rigidly on or relative to the support structure, in particular formed integrally in the support structure, and the adjusting means is in the form of a rotatable screw gear with a rotary handle for manual actuation, which by rotation adjusts a position of the first coupling structure, preferably with the convex surface, relative to the support structure and thus also relative to the second and third coupling structures. In other words, the relative position of the first coupling structure, in particular the convex surface, is adjustable by means of a rotary actuation of a rotary handle which is arranged on the adjusting means. In particular, the first coupling structure is engaged with the adjusting means via a threaded connection, as a result of which the coupling structure can be displaced or is displaced axially through the thread by a rotary movement of the rotary handle. Such a design with a screw gear and an operable rotary handle ensures the most precise fine adjustment possible. In particular, such a thread (compared to a spring or a clamping lever) compensates for tolerances that may arise due to the sterile cover above it. The support structure of this fastening adapter in this embodiment is designed, for example, in the form of a fork with a second and third coupling structure, which are each arranged on the fork arms or alternatively, in a further embodiment, has a closed (circumferential) contour, on the inner circumferential contour of which, in particular on one side, the second and third coupling structure are arranged. The support structure of this alternative design with a closed contour is formed in such a way that it encompasses the entire circumference of a fastening section.

According to a further embodiment, the screw gear has a torque limiter which is rigidly connected to the rotary handle and which only transmits the limit torque when a limit torque is exceeded and also provides an operator with haptic and/or acoustic feedback. In other words, an (additional) torque limiter, which is preferably located inside the screw gear, limits the torque that is applied to the rotary handle by the operator. Preferably, if a limit torque is exceeded, a spinning of the screw gear is initiated, whereby no further screwing process and therefore no further translational movement of the first coupling structure is possible. This prevents the adjusting means from being overtightened, which could lead to damage to the sterile barrier or the fastening adapter and/or fastening section components themselves. Alternatively or additionally, the torque limiter enables a haptic and/or acoustic signal to be emitted to the operator so that the operator recognizes when a limit torque has been reached. For example, when the limit torque is exceeded, a click can be emitted as an acoustic signal as well as a haptic signal, similar to a torque wrench.

In particular, the fastening adapter can be sterile-packed in sterile packaging so that after opening the sterile packaging it can be coupled directly to the fastening section (with interposed sterile cover) and fastened using the adjusting means.

According to a further embodiment, the support structure of the fastening adapter can have a guide stop (as a type of limiting edge) in the form of a projection, which is formed in the region of the first coupling structure and in particular projects in the same direction as the first coupling structure and lies parallel to the plane of the first second and third coupling structure, in order to geometrically align the fastening adapter in the first state of the first relative positioning with respect to a fastening section by means of the guide stop. Such a guide stop ensures that the fastening adapter and in particular the first coupling structure can be guided and arranged in a correct position or alignment to the fastening section during the coupling process. This prevents incorrect, especially skewed, alignment of the fastening adapter.

In a further advantageous embodiment of the disclosure, the first, second, and third coupling structures lie in a plane, the support structure has a first arm with a first coupling structure and a second arm with a second coupling structure and the first coupling structure lies opposite the second coupling structure and the first and/or the second arm are designed such that they can be elastically deflected relative to each other to enable relative positioning of the first coupling structure with respect to the second coupling structure, and the adjusting means has a hinge-like lever pull/snap lever with a form-fitting/stretchable (as bendable but not stretchable in the longitudinal direction due to the small thickness of the steel band) steel band, in particular further comprising two clamping jaws on the steel band, which connects the two arms to each other and forms a closed circumferential contour together with the arms and a shortening of the steel band between two points is adjustable via the hinge-like lever pull, resulting in an elastic deformation of the first and second arms towards each other in order to reduce a relative distance of the first coupling structure to the second coupling structure and to fix the fastening adapter in the second state of the second relative positioning to the fastening section. In other words, the fastening adapter preferably has two (at least slightly) flexible/elastic arms which can be moved relative to one another in a predetermined range by applying force and each have a lateral coupling structure, in particular in the form of a curved projection. The arms are fastened with a flexible steel band/steel plate, which is arranged on the outer contour of the fastening adapter and connects the two arms at the front and which can be shortened using a hinge-like lever as an adjusting means. The shortening or contraction of the steel strip also requires, in particular, the pressing of sliding jaw elements, which are further applied to the fastening interface by actuating the lever. This narrows the circumference of the fastening adapter, which causes the adapter to grip tightly around the fastening section of the medical robot and places the fastening adapter in the second state of the second relative positioning of the coupling structures. The fastening adapter has a third coupling structure, which is arranged in particular on the inner contour side of the fastening adapter opposite the steel strip, in particular with the clamping jaws.

According to a further embodiment, the fastening adapter has the instrument interface for the connection/mounting of a sterile medical product, in particular an instrument, as well as two alignment elements or projections which are arranged on the inner contour side which has the third coupling structure and are intended to prevent lateral displacement of the fastening adapter during a mounting process.

In a further preferred aspect of the disclosure, the fastening adapter, in particular the support structure, has a (high-performance) plastic, preferably PA 66 GRP or PEEK GRP, as material, in particular the support structure as a whole is made of this material. This ensures that no electrical contact is made between the fastening adapter and the medical robot. In particular, the support structure of the fastening adapter can be made of a mechanically resilient or stronger material, in particular metal as a material, preferably stainless steel or a titanium alloy, and the electrical insulation can be realized via a (separate) insulation sleeve (preferably made of PEEK), in particular at the front instrument interface.

The present disclosure further relates to a medical fastening system for releasably fastening a fastening adapter to a fastening section of a medical robot, in particular a robot-guided surgical microscope, which is arranged in particular within a sterile cover as a sterile barrier. The fastening system has a separate fastening adapter according to the disclosure and the fastening section has at least a first, second, and third counter-coupling structure, which are (in each case) designed to be form-fitting and complementary to or with the first coupling structure, the second coupling structure and the third coupling structure of the fastening adapter and interact. In other words, the fastening section, with which the fastening adapter can be coupled, has corresponding (complementary) counter-coupling structures which are (geometrically) designed in such a way that the respective coupling structures of the fastening adapter engage geometrically and positively in them during the coupling process. The coupling structures and counter-coupling structures of the fastening adapter and fastening section are therefore specially matched to each other.

In particular, according to an aspect of the present disclosure, a set of a first fastening adapter according to the present disclosure and an associated first fastening section and a different second fastening adapter according to the present disclosure with an associated second fastening section may be provided, which are not interchangeable with each other, wherein the first and second fastening sections are mounted or mountable on the robot so that only the first fastening adapter can be coupled to the first fastening section and only the second fastening adapter can be coupled to the second fastening section in order to prevent confusion by a healthcare professional. In this way, a set can be provided that enables a defined local assignment of the fastening adapter (to the associated complementary fastening section) without accidental replacement.

In a (particularly first) advantageous embodiment, the fastening section of the medical fastening system has a plate-shaped base structure with a central opening perpendicular to the plane of the base structure (for example to enable a surgical microscope to be viewed through this opening) and, on a front side (i.e., a side surface of the plate-shaped base structure), a first counter-coupling structure with a concave notch, which is aligned in the front direction and whose alignment lies in the plane of the base structure, and has, on a side region lateral to the central opening, a second and a third counter-coupling structure with a concave notch, which are each aligned in the opposite direction to the front direction, so that in the case of the fastening adapter with the first, the second and the third coupling structure, which lie in one plane and each have a projecting/protruding convex surface, this convex surface interacts in each case with the concave notch in order to fix a position of the fastening adapter relative to the fastening section by means of positive locking of the at least three contact points. In other words, a central (through) opening is arranged on the plate-shaped base structure of the fastening section, in which the medical robot, in particular the surgical microscope, can be arranged. The fastening section has three concave counter-coupling structures that are designed in such a way that they accommodate the respective coupling structures of the fastening adapter in a complementary manner during a coupling process.

In a further (in particular second) advantageous embodiment, the fastening section of the medical fastening system has a plate-shaped base structure with a central opening perpendicular to the plane of the base structure (or the longitudinal axis of the opening is perpendicular to the plate-shaped base structure) and, on a front side, a first counter-coupling structure with a concave notch, which is aligned in the front direction (longitudinal axis of the concave notch is aligned in the front direction) and lies in the plane of the base structure, and, on a rear side, which is arranged opposite the front side of the base structure, a second and a third counter-coupling structure, each with a concave notch, which are each aligned opposite the front direction, so that in the case of the fastening adapter with the first, the second, and the third coupling structure, which lie in one plane and each have a protruding/projecting convex surface, this convex surface interacts in each case with the concave notch in order to fix a position of the fastening adapter relative to the fastening section by means of positive locking (via the relative movement by the adjusting means) of the at least three contact points.

In particular, the second and third counter-coupling structures are arranged next to each other on the rear side of the base structure, which is opposite the front side with the first counter-coupling structure. In other words, in this embodiment, all three counter-coupling structures are arranged on the base structure in such a way that the coupling structures of the fastening adapter of this embodiment, which are arranged along the closed inner contour of the fastening adapter, can engage or grip into it in a complementary manner.

In the further (in particular third) advantageous embodiment, the fastening section has a plate-shaped base structure with a central opening perpendicular to the plane of the base structure and has a first counter-coupling structure and a second counter-coupling structure arranged on two opposite side walls and a third counter-coupling structure on a front side, so that in the fastening adapter with the first arm with the first coupling structure and the second arm with the second coupling structure, which are designed such that they can be deflected elastically relative to one another, these first, second, and third coupling structures interact with the respective counter-coupling structure, in particular in interaction with the two clamping jaws on the steel band which connects the two arms to one another, in order to fix a position of the fastening adapter relative to the fastening section by means of positive locking of the at least three contact points.

In a further advantageous aspect of the disclosure, the medical fastening system comprises a separate sterile cover arranged or arrangeable between the fastening adapter and the fastening section and in contact with at least the first, second, and third counter-coupling structure of the fastening section and the first, second, and third coupling structure of the fastening adapter. In other words, a sterile cover/sterile barrier, in particular in the form of a sterile plastic film/cover, is arranged between the sterile fastening adapter and the (non-sterile) fastening section of the medical robot, whereby the sterility of the medical instrument to be coupled is not impaired/influenced by the mounting/coupling to the medical robot via the fastening adapter. In particular, the medical fastening system is designed in such a way that the sterile cover can be easily positioned or clamped between the coupling structures of the fastening adapter and the complementary counter-coupling structures of the fastening section without any risk of damage or breaking through the sterile barrier. In particular, the fastening adapter only has rounded surfaces and (round) “edges” on the coupling structures, which engage in the counter-coupling structures indirectly via the sterile cover, so as not to damage the sterile cover.

The present disclosure further relates to a medical device, in particular a medical robot, for example in the form of a robot-guided surgical microscope, with a sterile cover for fastening/coupling sterile medical products to the medical device. The medical device, in particular the robot, has at least one fastening section and at least one fastening adapter according to the disclosure, in particular a plurality of fastening sections and fastening adapters, for example as a fastening adapter set, or the medical device, in particular the robot, has a fastening system according to the disclosure (with fastening section and fastening adapter according to the present disclosure). The sterile cover is arranged between the fastening section and the fastening adapter fastened to it in such a way that the robot with the fastening section(s) is/are arranged in a non-sterile area and the fastening adapter with, for example, a medical instrument is arranged in a sterile area and thus separated from each other.

Any disclosure related to the fastening adapter of the present disclosure also applies to the fastening system of the present disclosure as well as vice versa.

### BRIEF DESCRIPTION OF THE DRAWINGS

The disclosure is explained in more detail below with reference to advantageous embodiments and with reference to the associated figures.

FIG. 1 shows a perspective (top) view of a partial section of a medical fastening system according to a first advantageous embodiment of the disclosure;

FIG. 2 shows a plan view of a sectional view through a plane of the medical fastening system according to the first advantageous embodiment of the disclosure, which is spanned by the three coupling structures of the fastening adapter;

FIG. 3 shows a perspective view of the medical fastening system according to the first advantageous embodiment in a state coupled to a surgical microscope and thus also a medical device in the form of a robot according to a preferred embodiment;

FIG. 4 shows a perspective view of a fastening adapter according to a first advantageous embodiment;

FIG. 5 shows a perspective front view of a fastening section according to a first advantageous embodiment of the disclosure for a medical robot;

FIG. 6 shows a further perspective rear view of the fastening section according to the first advantageous embodiment of the disclosure;

FIG. 7 shows a longitudinal sectional side view through a partial section of the fastening adapter according to the first embodiment in the coupled state with the fastening section according to the first embodiment of the disclosure;

FIG. 8 shows a longitudinal sectional side view through the adjusting means of the fastening adapter according to the first advantageous embodiment of the disclosure, in a decoupled state of the fastening adapter;

FIG. 9 shows a further longitudinal sectional side view through the adjusting means of the fastening adapter according to the first advantageous embodiment of the disclosure, in a coupled state of the fastening adapter;

FIG. 10 shows a perspective (side) view of a medical fastening system according to a second advantageous embodiment of the disclosure;

FIG. 11 shows a perspective view of the medical fastening adapter according to a second advantageous embodiment of the disclosure;

FIG. 12 shows a perspective longitudinal sectional view through the adjusting means of the fastening adapter according to the second advantageous embodiment of the disclosure, in a coupled state of the fastening adapter;

FIG. 13 shows a perspective longitudinal sectional side view through one of the second or third coupling structure of the fastening adapter according to the second embodiment of the disclosure, in a coupled state of the fastening adapter;

FIG. 14 shows a perspective side view of the medical fastening system according to the second advantageous embodiment in a state coupled with a surgical microscope;

FIG. 15 shows a longitudinal sectional side view through the adjusting means/fastener for the first or the second embodiment of the medical fastening adapter in an alternative embodiment;

FIG. 16 shows a perspective top view of a medical fastening system according to a third advantageous embodiment of the disclosure;

FIG. 17 shows a perspective view of the medical fastening adapter according to a third advantageous embodiment of the disclosure;

FIG. 18 shows a further perspective view of the medical fastening adapter according to the third advantageous embodiment of the disclosure;

FIG. 19 shows a perspective (front) view of the fastening section according to a third advantageous embodiment of the disclosure;

FIG. 20 shows a perspective (side) view of the fastening section according to a third advantageous embodiment of the disclosure;

FIG. 21 shows a perspective side view of the medical fastening system according to the third embodiment during a coupling process;

FIG. 22 shows a longitudinal sectional side view through an alignment element of the fastening adapter according to the third embodiment during a coupling process.

The figures are merely schematic in nature and are provided solely for the purpose of understanding the present disclosure. It should be noted that the features of the individual embodiments are interchangeable and may occur in a particular combination.

### DETAILED DESCRIPTION

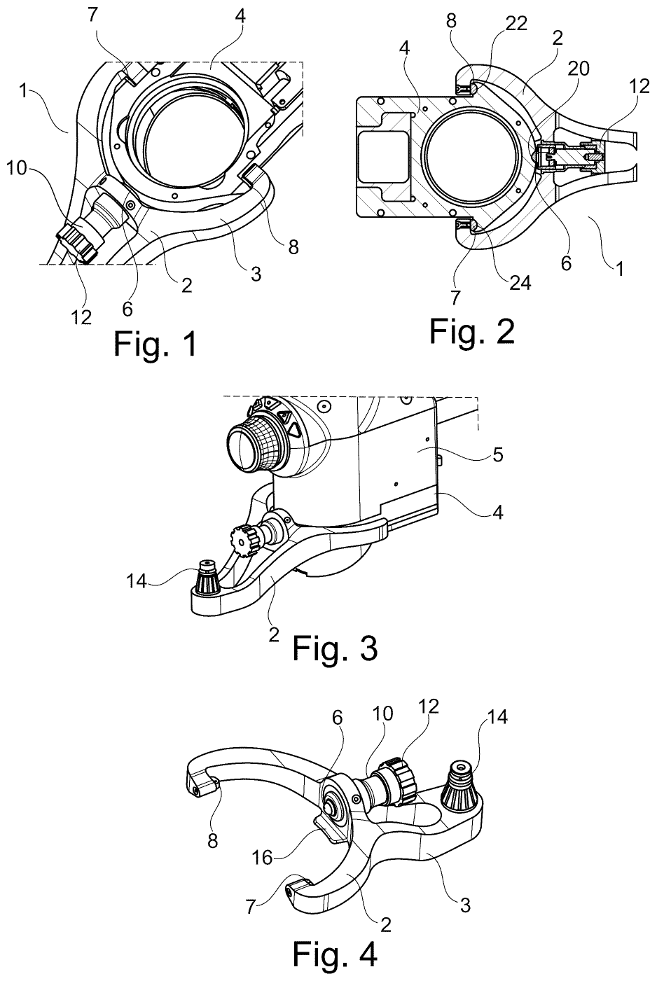

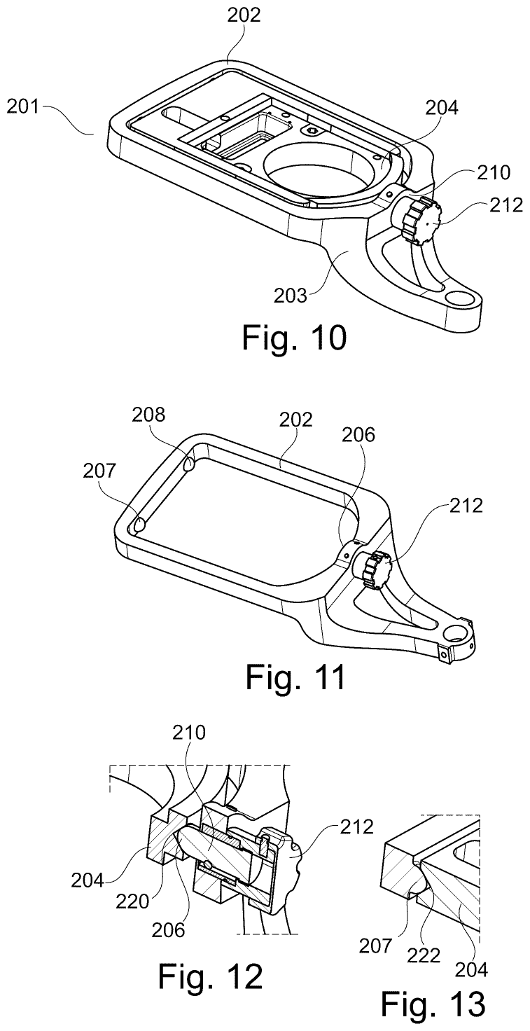

In the following, the present disclosure is described with reference to the advantageous embodiments with reference to FIGS. 1 through 22. FIGS. 1 through 9 show views of a fastening system according to a first preferred embodiment comprising a fastening adapter of a first preferred embodiment and an (associated) fastening section of a first preferred embodiment for a medical robot according to a preferred embodiment. FIGS. 10 through 15 further show a fastening system according to a further, second preferred embodiment with a fastening adapter and a fastening section, each according to a second preferred embodiment. FIGS. 16 through 22 finally show a fastening system according to a further, third preferred embodiment with a fastening adapter and a fastening section each according to a third preferred embodiment.

FIG. 1 shows a perspective view of a partial section of a fastening system 1 and FIG. 2 a sectional view through a plane which is spanned by three coupling structures 6, 7, 8 of a fastening adapter 2, according to the first advantageous embodiment of the medical fastening system 1. The fastening adapter 2 according to the first advantageous embodiment has a support structure 3, in particular a fork-shaped structure. The fastening adapter 2 is shown in a coupled state with a (separate) fastening section 4, which is designed as part of a medical robot 5 or can be connected to it. The coupling or contact between the components of the fastening adapter 2 and the fastening section 4 takes place via a first coupling structure 6, a second coupling structure 7 and a third coupling structure 8 of the fastening adapter 2, which in this first embodiment are designed as convex/curved surfaces in the form of spherical hemispheres. These three coupling structures 6, 7, 8 are arranged in one plane and engage in three complementary counter-structures 20, 22, 24 of the fastening section 4, which in this first embodiment are designed as (complementary) concave indentations (for example with recessed hemispherical surfaces). The first coupling structure 6 is arranged on a central axis of the fastening adapter 2 and is directed against the second coupling structure 7 and the third coupling structure 8. It can also be said that the first coupling structure 6 with the convex surface is opposite the second and third coupling structures, and in this embodiment a longitudinal axis of the first coupling structure 6 is parallel to the longitudinal axes of the second and third coupling structures 7, 8, wherein the directions of the convex surfaces are opposite.

The second and third coupling structures 7, 8 are arranged at the end sections of the fork-like arms of the support structure 3 of the fastening adapter 2, in particular along an imaginary common axis, and are at the same time laterally spaced from the first coupling structure 6. Furthermore, the fastening adapter 2 has an adjusting means 10 in the form of a screw gear, which can be operated manually via a rotary handle 12. This adjusting means 10 is in connection with the first coupling structure, whereby the first coupling structure 6 can be displaced translationally/axially along the central axis of the fastening adapter in the direction of the second and third coupling structures 7, 8 or away from them when the rotary handle 12 is turned manually. In this way, the fastening adapter can be transferred from a first state of a first relative positioning of the coupling structures 6, 7, 8 relative to one another to a second state of a second geometric relative positioning by means of the adjusting means 10, as also explained in detail below.

FIG. 3 shows a perspective side view of the medical fastening system 1 according to the first advantageous embodiment in a state coupled with a robot-guided surgical microscope 5. FIG. 3 thus also shows the medical robot according to a preferred embodiment. The fastening section 4 can be designed as an integral part of the surgical microscope 5 or alternatively, as in the present case, can be mounted as a separate component of a fastening section 4 in an additional step on this robot-guided surgical microscope 5. FIG. 3 also shows a cylindrical or conical instrument interface 14 for a sterile medical instrument. In the coupled state of the fastening adapter 2, the instrument interface 14 is perpendicular to a plane of the fastening system 1 or a plane of the fastening adapter (i.e., parallel to an axis of a central opening) and is arranged on the end section of the support structure 3 which faces the fork-shaped arms of the support structure 3. The support structure 3 can also be described as Y-shaped with two opposing arms, wherein the instrument interface 14 is arranged on the section facing away from the arms (the lower part of the Y, so to speak). In a side view, the support structure 3 also does not lie entirely in one plane, but the arms with the second and third coupling structures 7, 8 and the adjusting means 10 with the first coupling structure 6 lie essentially in one plane, which is adjoined by a sloping section with a distal front section with the instrument interface 14, wherein the front section is again offset parallel to the plane of the arms (in the manner of a step in order to ensure accessibility to the adjusting means 10).

FIG. 4 shows a perspective view of the fastening adapter 2 according to the first advantageous embodiment. As described above, the support structure 3 of the fastening adapter of this first embodiment has a fork-like shape with two arms. One of the second and third coupling structures 7, 8 is arranged at the end section of each of these arms. The first coupling structure 6, which is connected to the adjusting means 10, is arranged in the middle of the support structure 3. The adjusting means 10 has a rotary handle 12, by means of which the first coupling structure 6 can be axially displaced (rotation is converted into translation). One end section of the support structure 3 has a conical (instrument) interface 14 for the coupling of a medical instrument as a medical product. In addition, the support structure 3 has a projecting guide stop 16 below the first coupling structure 6, which is formed in one piece with the support structure 3 and is intended to geometrically align the fastening adapter 2 with respect to the fastening section 4. The guide stop 16 protrudes in the same direction as the first coupling structure 6.

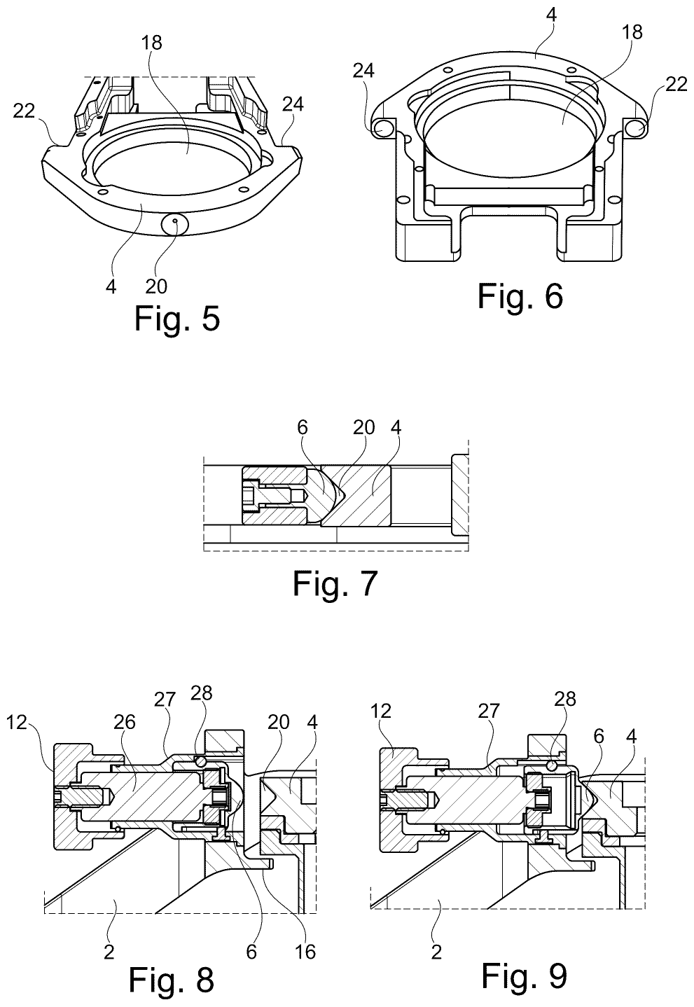

FIG. 5 shows a perspective front view of a fastening section 4 and FIG. 6 shows a perspective rear view of the fastening section 4 according to a first advantageous embodiment of the disclosure for a medical robot.

The fastening section 4 according to this first embodiment has a plate-shaped base structure with a central opening 18 perpendicular to the plane of the base structure. The opening 18 is intended in particular to accommodate a lens of a medical surgical microscope 5 (centrally). On its front side, the base structure has a first negative feedback structure 20 with a concave notch that is aligned in the front direction and lies in the plane of the base structure. The two averted side walls of the base structure each have a projection in the lateral direction, on which the second and third counter-coupling structures 22, 24 are each integrated with a concave notch, which are each aligned in the opposite direction to the first counter-coupling structure 20 (their virtual longitudinal axes, starting from the notch, point away from the first counter-coupling structure). The first, second, and third counter-coupling structures 20, 22, 24 lie in the same plane and are designed and adapted to receive the coupling structures 6, 7, 8 of the fastening adapter 2 in a complementary manner in order to interact with them and to fix a position of the fastening adapter 2 relative to the fastening section 4 by means of positive locking of the at least three contact points in the second state of the second relative positioning.

FIG. 7 shows a longitudinal sectional side view through a partial section of the fastening adapter 2 according to the first embodiment in the coupled state with the fastening section 4 according to the first embodiment of the disclosure. In this view, the hemispherical first coupling structure 6 of the fastening adapter 2, which is firmly connected to the adjusting means 10 not shown here, preferably via a screw connection, engages in the counter-coupling structure 20 of the fastening section 4 provided for this purpose. FIG. 7 thus shows a coupled state of the fastening adapter 2 and the fastening section 4.

FIG. 8 shows a longitudinal sectional side view through the adjusting means 10 of the fastening adapter 2 according to the first advantageous embodiment of the disclosure, in a first decoupled state of the fastening adapter 2 and FIG. 9 shows the same longitudinal sectional side view in a second coupled state of the fastening adapter 2. In this embodiment, the adjusting means 10 is in the form of a screw gear with a rotary handle 12. By manually turning the rotary handle 12, the first hemispherical coupling structure 6 is moved axially in the direction of the first counter-coupling structure 20 provided on the fastening section 4 and ultimately brought into positive (active) engagement. The adjusting means 10 in this first embodiment of the fastening adapter 2 has a torque limiter 26, which is firmly or rigidly connected to the rotary handle 12, in particular via a screw connection. The torque limiter 26 ensures that only the limit torque is transmitted if a limit torque is exceeded and also provides an operator with haptic and/or acoustic feedback/signal. Furthermore, a fixing ball 28 is arranged in an eccentric axial groove within a sleeve 27 of the screw thread and the fixing ball 28 simultaneously engages in a spherical notch/milling on the outer circumference of the first coupling structure 6. This prevents rotation of the first coupling structure 6 together with the rotary handle 12. Thus, the fixing ball 28 moves together with the first coupling structure 6 in the direction of the counter-coupling structure 20 when the rotary handle 12 is actuated for a coupling operation of the fastening adapter 2. A purely axial displacement of the first coupling structure 6 ensures that an existing sterile cover, which is clamped between the first coupling structure 6 and the first counter-coupling structure 20, is wound up or destroyed.

FIG. 10 shows a perspective view of the medical fastening system 201 according to a second advantageous embodiment of the disclosure. The fastening adapter 202 in this second embodiment has a support structure 203 with a closed contour (in contrast to the two arms as in the first embodiment). This support structure 203 completely surrounds the outer contour of the fastening section 204 of this second embodiment. Likewise, the fastening adapter 202 of this second embodiment has a fastener 210 with a rotary handle 212, similar to the fastener 10 of the first embodiment of the fastening adapter 2.

FIG. 11 shows a perspective view of the medical fastening adapter 210 according to the second advantageous embodiment of the disclosure. The support structure 203 of the fastening adapter 202 has a closed contour for enclosing the outer contour of a fastening section 204. The first coupling structure 206 is connected to the adjusting means 210, which can be actuated via the rotary handle 212, and points into the interior of the closed contour of the support structure. The second coupling structure 207 and the third coupling structure 208 are arranged on the inner side, which is opposite the side with the first coupling structure 206. An imaginary axis of the first coupling structure 206 extends through the center axis of the fastening adapter 202 and the second and third coupling structures 207, 208 are arranged on imaginary axes which are each spaced at an equal distance, parallel, from the imaginary axis of the first coupling structure 206. The first coupling structure 206 also points in the opposite direction to that in which the second and third coupling structures 207, 208 point, and thus towards each other.

FIG. 12 shows a perspective longitudinal side view through the adjusting means 210 of the fastening adapter 202 according to the second advantageous embodiment in a coupled state of the fastening adapter 210, and FIG. 13 shows a perspective longitudinal side view through one of the second or third coupling structure 207, 208 of the fastening adapter 202 according to this second embodiment in a coupled state of the fastening adapter 202. The adjusting means/fastener 210 of this second embodiment is also designed in particular in the same way as the fastener 10 of the first embodiment. FIG. 12 shows the engagement of the first coupling structure 206, which is rigidly connected to the fastener 210, with the complementary counter-coupling structure 220 of the fastening section 204. FIG. 13 shows the positive engagement of the second coupling structure 207 with the complementary counter-coupling structure 222 of the fastening section 204.

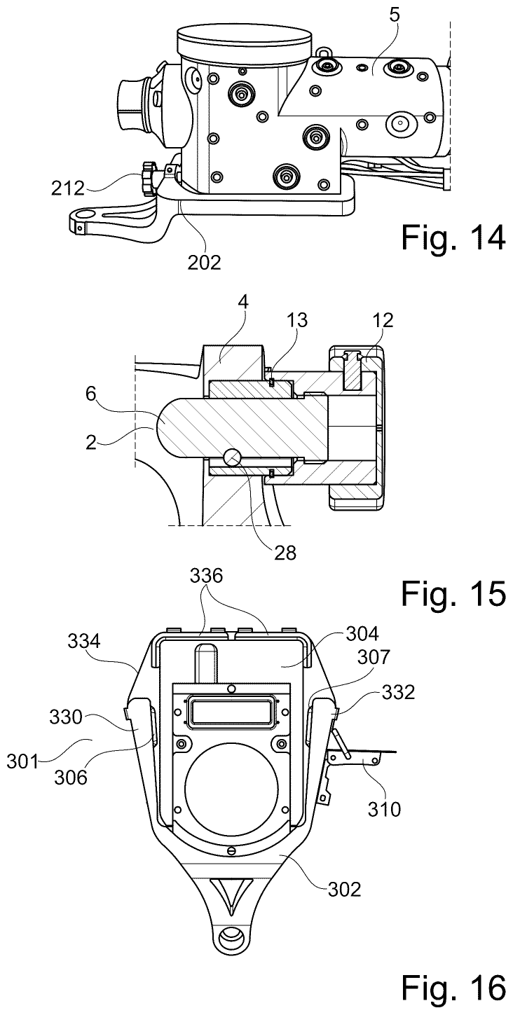

FIG. 14 shows a perspective side view of the medical fastening system 201 according to the second advantageous embodiment in a state coupled to a robot-guided surgical microscope 5 (as a medical robot according to the present disclosure). The support structure 203 of the fastening adapter 202 completely embraces a fastening section 204, which is arranged in a section of the surgical microscope 5. The sterile cover preferably arranged between the surgical microscope 5, in particular the fastening section 204, and the fastening adapter 202 is not shown in FIG. 14.

FIG. 15 shows a longitudinal side view through an alternative embodiment of the fastener 10 for the first (or the second) embodiment of the medical fastening adapter 2. This alternative embodiment of the fastener 10 does not have a torque limiter 26. The sleeves of the fastener 10 are connected here via a snap ring 13. Furthermore, this design also has the fixing ball 28, which prevents the first coupling structure 6 from twisting.

FIG. 16 shows a perspective top view of the medical fastening system 301 according to a third advantageous embodiment of the disclosure. The fastening adapter 302 in this third embodiment has a first arm 330 and a second arm 332 arranged opposite it, which are elastically formed. In this illustration, the fastening adapter 302 is already aligned with the fastening section 304, but is not yet coupled. The coupling sometimes takes place via an engagement of the first coupling structure 306 and the second coupling structure 307 in the respective counter-coupling structures 322, 324 (not shown here) of the fastening section 304. Furthermore, the fastening adapter 302 is additionally held by two clamping jaws 336, which are connected to a flexible/shortenable steel band 334. These clamping jaws 336 are adapted to the outer contour of the fastening section 304. In this embodiment, the fastener has a hinge-like lever pull 310 (in FIG. 16 still in a first uncouplable and couplable state). Actuation of the lever pull 310 causes the first and second resilient arms 330, 332 and thus also the first coupling structure 306 and the second coupling structure 307 to deflect relative to one another and causes the steel band 334 to shorten, thereby fixing a position of the fastening adapter 302 relative to the fastening section 304 or coupling the two components. The lever pull 310 fastened to one arm thus causes a contraction of the steel band fastened to the other, opposite arm with accompanying contraction and elastic deflection of the arms towards each other, whereby a relative distance between the first and second coupling structure is shortened (i.e., from a first relative positioning towards a second relative positioning).

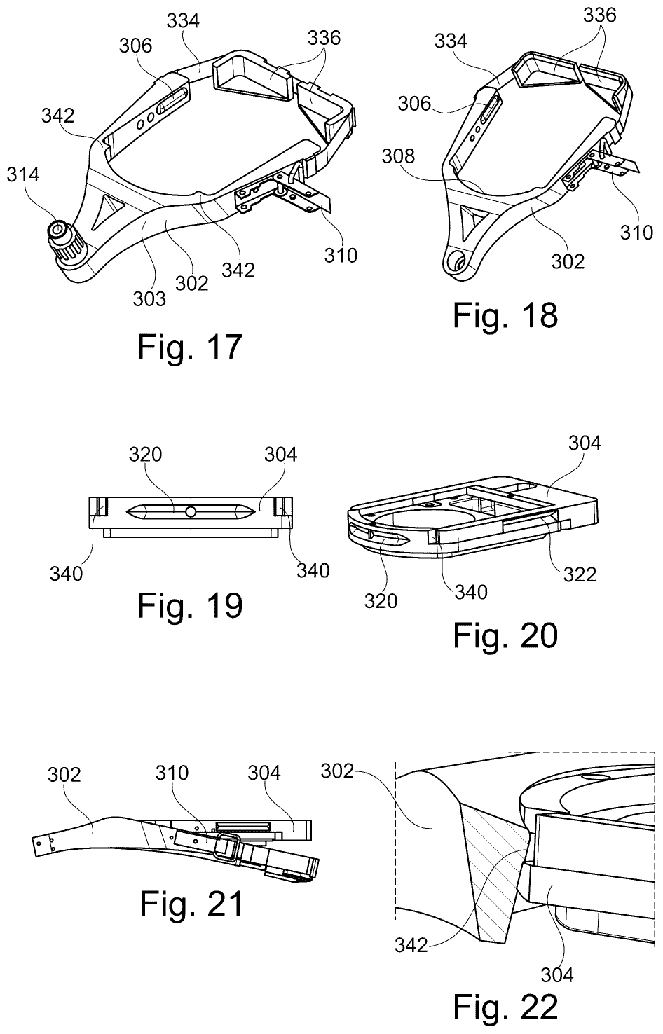

FIG. 17 and FIG. 18 each show a perspective side view of the medical fastening adapter 302 according to the third advantageous embodiment of the disclosure. The support structure 303 of the fastening adapter 302 has a conical/cylindrical instrument point 314 at one end section for the connection of a medical instrument (not shown in FIG. 18). Furthermore, the support structure 303 of the fastening adapter 302 has a third coupling structure 308 (not shown here) on an inner side, which is arranged opposite the clamping jaws 336. The first, second, and third coupling structures 306, 307, 308 are preferably shaped as elongated, in particular stadium-shaped, protrusions. Furthermore, the support structure 303 has two projection-like alignment elements 342, which are arranged on the inner contour side and lie opposite the clamping jaws 336 as viewed in the longitudinal direction. These alignment elements 342 are designed to prevent lateral displacement of the fastening adapter 302 during a mounting/coupling operation to the fastening section 304.

FIG. 19 shows a perspective front view and FIG. 20 a perspective side view of the fastening section 304 according to a third advantageous embodiment of the disclosure. The third counter-coupling structure 320 is arranged on the front side of the fastening section 304 and has an elongate, in particular stadium-shaped, notch as a geometry into which the third coupling structure 308 of the fastening adapter 302 can engage in a complementary manner. The first and second counter-coupling structures 322, 324 are arranged on the side walls of the fastening section 304, which preferably form a rectangular recess on the side walls. In addition, indentations 340 are provided at the respective corners of the fastening section 304, which are arranged between the side walls and the front side, each of which receives an alignment element 342 of the fastening adapter 302 in a complementary manner during a coupling process.

FIG. 21 shows a perspective side view of the medical fastening system 301 and FIG. 22 shows a longitudinal side view through an alignment element 342 of the fastening adapter 302 according to the third embodiment during a coupling process. It can be seen from FIG. 21 that a coupling or mounting process of the fastening adapter 302 to the fastening section 304 first begins with an alignment of the third coupling structure 308 with the associated counter-coupling structure 320 and the two alignment elements 342 with the associated indentations 340 on the fastening section 304. The opposite end section of the fastening adapter 302, on which the clamping jaws 336 are arranged, is angled with respect to the fastening section 304 and is pulled over it at an angle, so to speak. This is possible because the steel band 334 with the clamping jaws 336 is in the loosened or extended state, whereby in this state the inner circumference of the fastening adapter 302 is larger than the outer circumference of the fastening section 304. Only after the lever pull 310 is actuated does the steel band 334 with the clamping jaws 336 shorten and at the same time the first arm 330 and the second arm 332 are deflected towards each other, whereby the fastening adapter 302 is coupled to the fastening section 304.

### LIST OF REFERENCE SIGNS

1, 201, 301 medical fastening system
2, 202, 302 medical fastening adapter
3, 203, 303 support structure
4, 204, 304 fastening section
5 medical robot, surgical microscope
6, 206, 306 first coupling structure
7, 207, 307 second coupling structure
8, 208, 308 third coupling structure
10, 210 screw gear
12, 212 rotary handle
13 snap ring
14, 314 instrument interface
16 guide stop
18 opening
20, 220, 320 first negative feedback structure
22, 222, 322 second negative feedback structure
24, 224, 324 third negative feedback structure
26 torque limiter
27 sleeve
28 fixing ball
310 lever pull
330 first arm
332 second arm
334 steel strap
336 clamping jaw
340 notch
342 alignment element

1. A medical fastening adapter for detachable fastening to a fastening section of a medical robot, the medical fastening adapter comprising:
a support structure with an instrument interface;
at least a first coupling structure, a second coupling structure, and a third coupling structure that are each designed for a positive connection to the fastening section; and
an adjusting element adapted to adjust positions of the first coupling structure, the second coupling structure, and the third coupling structure relative to each other in such a way that, in a first relative positioning, the medical fastening adapter is couplable to the fastening section, and in a second relative positioning, the medical fastening adapter is fixed to the fastening section, a position of the medical fastening adapter relative to the fastening section being determined by the first coupling structure, the second coupling structure, and the third coupling structure.

2. The medical fastening adapter according to claim 1, wherein the first coupling structure, the second coupling structure, and the third coupling structure lie in one plane and each have a convex surface, wherein at least the convex surface of the first coupling structure faces the convex surface of the second coupling structure and/or the third coupling structure, and the convex surface of the first coupling structure is displaceable in translation via the adjusting element in order to change a relative distance.

3. The medical fastening adapter according to claim 2, wherein the second coupling structure and the third coupling structure are rigidly arranged on the support structure, and the adjusting element is designed as a rotatable screw gear with a rotary handle for manual actuation, which by rotation adjusts a position of the convex surface of the first coupling structure relative to the support structure and thus also relative to the second coupling structure and the third coupling structure.

4. The medical fastening adapter according to claim 3, wherein the rotatable screw gear has a torque limiter rigidly connected to the rotary handle and which, when a limit torque is exceeded, transmits only the limit torque and also provides an operator with haptic and/or acoustic feedback.

5. The medical fastening adapter according to claim 2, wherein the support structure of the medical fastening adapter has a guide stop forming a projection which is formed in a region of the first coupling structure and projects in a same direction as the first coupling structure and is parallel to a plane of the first coupling structure, the second coupling structure, and the third coupling structure in order to geometrically align the medical fastening adapter with respect to the fastening section via the guide stop in the first relative positioning.

6. The medical fastening adapter according to claim 1, wherein the first coupling structure, the second coupling structure, and the third coupling structure lie in one plane, the support structure having a first arm with the first coupling structure and a second arm with the second coupling structure, wherein the first coupling structure lies opposite the second coupling structure and the first arm and/or the second arm is elastically deflectable, and the adjusting element comprises a hinge-like lever pull with a steel band, which connects the first arm and the second arm to each other and a shortening of the steel band between two points is adjustable via the hinge-like lever pull, which results in an elastic deformation of the first arm and the second arm towards each other to reduce a relative distance of the first coupling structure to the second coupling structure and to fix the medical fastening adapter to the fastening section in the second relative positioning.

7. The medical fastening adapter according to claim 1, wherein the medical fastening adapter comprises a plastic.

8. A medical fastening system for detachable fastening of a fastening adapter to a medical robot, the medical fastening system comprising:
the medical fastening adapter according to claim 1; and
a fastening section on the medical robot,
the fastening section comprising at least a first counter-coupling structure, a second counter-coupling structure, and a third counter-coupling structure that interact positively and complementarily with the first coupling structure, the second coupling structure, and the third coupling structure, respectively.

9. The medical fastening system according to claim 8, wherein:
the fastening section has a plate-shaped base structure with a central opening perpendicular to a plane of the plate-shaped base structure,
the fastening section comprising, on a front side, a first counter-structure with a first concave notch which is aligned in a front direction,
the fastening section further comprising, on a side region lateral to the central opening, a second counter-structure and a third counter-structure,
the second counter-structure having a second concave notch and the third counter-structure having a third concave notch, the second concave notch and the third concave notch each aligned in a direction opposite to the front direction,
the first coupling structure having a first convex surface, the second coupling structure having a second convex surface, and the third coupling structure having a third convex surface,
the first coupling structure, the second coupling structure, and the third coupling structure lying in one plane,
the first convex surface, the second convex surface and the third convex surface interacting with the first concave notch, the second concave notch, and the third concave notch, respectively, to fix a position of the medical fastening adapter relative to the fastening section by positive locking.

10. The medical fastening system according to claim 8, wherein:
the fastening section has a plate-shaped base structure with a central opening perpendicular to a plane of the plate-shaped base structure,
the fastening section comprising a first counter-structure with a first concave notch on a front side, which is aligned in a frontal direction,
the fastening section further comprising, on a rear side, which is arranged opposite the front side, a second counter-structure having a second concave notch and a third counter-structure having a third concave notch, the second concave notch and the third concave notch each aligned in a direction opposite to the frontal direction,
the first coupling structure having a first convex surface, the second coupling structure having a second convex surface, and the third coupling structure having a third convex surface,
the first coupling structure, the second coupling structure, and the third coupling structure lying in one plane,
the first convex surface, the second convex surface and the third convex surface interacting with the first concave notch, the second concave notch, and the third concave notch, respectively, to fix a position of the medical fastening adapter relative to the fastening section by positive locking.

11. The medical fastening system according to claim 8, wherein:
the fastening section has a plate-shaped base structure with a central opening perpendicular to a plane of the plate-shaped base structure,
the fastening section comprising a first counter-coupling structure and a second counter-coupling structure arranged on two remote side walls,
the fastening section further comprising a third counter-coupling structure on a front side,
the medical fastening adapter comprising a first arm with the first coupling structure and a second arm with the second coupling structure,
the first arm and the second arm being elastically deflectable relative to one another,
the first coupling structure, the second coupling structure, and the third coupling structure interacting with the first counter-coupling structure, the second counter-coupling structure, and the third counter-coupling structure in order to fix a position of the medical fastening adapter relative to the fastening section by positive locking.

12. The medical fastening system according to claim 8, further comprising a separate sterile cover arranged between the medical fastening adapter and the fastening section, the separate sterile cover connected to at least the first counter-coupling structure, the second counter-coupling structure, and the third counter-coupling structure as well as the first coupling structure, the second coupling structure, and the third coupling structure.

13. A medical device comprising the medical fastening system according to claim 8.

14. A medical device comprising:
at least one medical fastening adapter according to claim 1;
at least one fastening section; and
a sterile cover,
the sterile cover being arranged between the at least one fastening section and the at least one medical fastening adapter, so that the at least one fastening section is positionable in a non-sterile area, and the at least one fastening adapter is positionable in a sterile area.
